(19) United States
(12) Patent Application Publication
(10) Pub. No.: US 2022/0042116 A1
(43) Pub. Date: Feb. 10, 2022

(54) METHODS FOR ASSESSING VIRAL CLEARANCE

(71) Applicant: Anthony P. Shuber, Northbridge, MA (US)

(72) Inventor: Anthony P. Shuber, Northbridge, MA (US)

(21) Appl. No.: 17/390,252

(22) Filed: Jul. 30, 2021

Related U.S. Application Data
(60) Provisional application No. 63/064,176, filed on Aug. 11, 2020.
(60) Provisional application No. 63/059,004, filed on Jul. 30, 2020.

(51) Int. Cl. C12Q 1/70

(52) U.S. Cl. CPC C12Q 1/70

(57) ABSTRACT

The invention provides methods for diagnosing viral infections and determining a status of the infection, including transmissibility of the infection.

### CROSS-REFERENCE TO RELATED APPLICATIONS

This application claims priority to, and the benefit of, U.S. Provisional Application No. 63/059,004, filed Jul. 30, 2020, and U.S. Provisional Application No. 63/064,176, filed on Aug. 11, 2020, the content of each of which is incorporated by reference herein in its entirety.

### TECHNICAL FIELD

The invention generally relates to diagnostic methods, and, more particularly, to methods for diagnosing viral infections and determining a status of the infection, including transmissibility of the infection.

### BACKGROUND

There is increasing concern about the spread of contagious diseases, whether these may be influenza, common colds, potentially lethal viruses, or microbial or viral diseases that are not even known or identified at this time. For example, the rapid spread of the severe acute respiratory syndrome coronavirus-2 (SARS-CoV-2), resulting in a global pandemic, has placed an emphasis on the criticality of early detection and understanding the transmissibility of such contagious diseases.

For many viral diseases (SARS, SARS-CoV-2, Middle East respiratory syndrome (MERS) coronavirus, influenza virus, Ebola virus, and Zika virus), it is well known that viral RNA can be detected long after the resolution of symptoms. For example, with the measles virus, viral RNA can still be detected six to eight weeks after the clearance of the infectious virus. Viral clearance generally refers to the time it takes for the resolution of symptoms associated with the viral disease to clear. At that point, the body's immune system and/or treatments have successfully resolved a patient's symptoms and, in some instances, removed evidence of the virus. The immune system can neutralize viruses by lysing their envelope or aggregating virus particles, wherein such processes prevent subsequent infection but do not eliminate nucleic acid, which degrades slowly over time.

Current tests are able to confirm viral infection via conventional molecular viral detection (i.e., detecting viral RNA from a sample). If viral RNA is present, then a patient is identified as testing positive for the viral infection, and if the viral RNA is not present, then the patient is identified as testing negative. While current tests are able to determine the presence of viral RNA and thereby identify a patient as testing positive, such tests and diagnostic methodologies are unable to determine whether the tested sample is infectious and transmissible.

### SUMMARY

The present invention provides methods for diagnosing viral infections and for determining the status of the infection. More specifically, the invention improves on conventional molecular viral detection by differentiating between intact viral RNA indicative of an active (and transmissible) infection and fragmented RNA indicative of viral clearance. In addition, the invention allows for quantifying an amount of RNA suspected to be associated with an active (i.e., transmissible) viral infection and/or quantifying RNA fragments associated with a cleared viral infection. The invention takes advantage of the fact that, as the immune system clears a viral infection, viral RNA is degraded. The degraded RNA fragments that are produced as a result of viral neutralization by the immune system are characteristically smaller than fragments obtained from an active viral infection. The invention takes advantage of that insight in order to produce a sensitive and specific diagnostic that distinguishes between patients who have been infected and are producing intact virus (even though they may be asymptomatic), and thus are contagious, and patients who have been infected but are not longer producing active virus and, therefore, are not contagious with respect to the virus.

The invention provides tailored diagnosis relevant to the health status of an individual patient, allowing clinicians to clear patients who have had an infection well before conventional molecular assays would indicate clearance. In a preferred embodiment, the invention provides for obtaining a sample from an individual suspected of having a viral infection. Viral RNA, if present, is extracted from the sample and reverse-transcribed into cDNA with primer pairs comprising a binding member. The cDNA is then amplified and fragment length is determined for subsequent determination of transmissibility. The binding member can be any convenient moiety (e.g., haptenated or biotinylated). The amplicons are bound to a solid support via the binding member and a transmissible infection is identified as a number of amplicons exceeding a predetermined threshold length. The amplicons are preferably labeled for detection (e.g., with a colorimetric label or other identifiable marker).

Ideally, cDNA is amplified using a plurality of primer pairs that are targeted to different regions along a contiguous length of the cDNA. Each primer pair (forward and reverse) will amplify through the length of cDNA that is present from a 5′ end to a 3′ end. Because the primer pairs are arrayed along a contiguous cDNA fragment (i.e., they are targeted against regions of the viral cDNA known to be contiguous), each primer pair will produce an amplicon of equal length if the viral RNA from which the cDNA was derived was intact. If the viral RNA has been degraded, most of the primer pairs will produce no amplicon and any amplicon that is produced will be substantially shorter than the full-length amplicon expected from the intact RNA-derived cDNA. Thus, by simply assessing whether pairs of primers produce amplicons of substantially equal length, it is possible to diagnose an active infection that is still in the virulent state, and thereby make a determination that the patient remains contagious with respect to the virus.

In one embodiment, the primers are directed to selected regions of the cDNA along the length of a reverse-transcribed cDNA strand from one end to the other, with different colors representing the series of tiled primers. Thus, colorimetric detection can be accomplished simply by observing (and possibly quantifying) color. For example, an intact (full-length or near full-length) sequence will result in a multi-color output (indicating a transmissible virus); whereas a single color or a predomination of a single color, indicates RNA that has been degraded as a result of the immune response to infection, indicating a less-transmissible infection.

In some embodiments, methods of the present invention utilize quantitative PCR (qPCR) in order to provide relative quantities of amplicons. By knowing relative quantities of amplicons, subsequent quantitative analysis can be performed for the determination of whether a given sample is exhibiting an active (i.e., transmissible) viral infection or a cleared viral infection. In particular, as a viral infection is cleared by the immune system or therapy, the number of intact (long) fragments, indicative of an active viral infection, decreases and the number of shorter fragments, indicative of viral neutralization, increases. Accordingly, during an active infection, primer pairs will produce fragments of equivalent length, whereas, upon neutralization, fragments will be smaller. As such, the greater the number of smaller fragments, the greater the likelihood that the viral infection is cleared and the patient can be deemed to be non-contagious.

Another use of the invention is to detect active (i.e., infectious) virus on environmental samples, including surfaces (e.g., doorknobs, elevator buttons, hand rails, shopping carts, face masks, etc.). The principle of the invention is the same in that detection of intact RNA on the surface is indicative of the presence of virus that is infective and detection of small viral RNA fragments is indicative of virus that has been killed or is otherwise incapable of causing an infection. Methods of the invention are also useful to detect active virus in aerosol samples or droplets. Aerosol samples can be obtained in air or, more preferably, via the expulsion of droplets with a cough or sneeze.

### BRIEF DESCRIPTION OF THE DRAWINGS

FIG. 1 diagrams a method of assessing transmissibility of a virus.

FIG. 2 shows a sample from a patient suspected of having a viral infection and loading of the sample into an instrument capable of performing one or more assays on the sample to determine whether viral RNA associated with the viral infection is present and the transmissibility of the viral infection.

FIG. 3 is a graph illustrating a process of viral clearance resulting in fragmentation of viral DNA.

FIGS. 4A and 4B illustrate operations of the transmissibility assessment method on a full-length viral RNA strand and a fragmented RNA strand, respectively.

FIGS. 5A and 5B are graphs illustrating quantitative analysis of the full-length and fragmented RNA strands, respectively, of FIGS. 4A and 4B having undergone qPCR.

FIG. 6 illustrates operations of converting mRNA to cDNA via reverse transcription in accordance with the methods of the present invention.

FIGS. 7A, 7B, and 7C show representative illustrations of populations of resulting RNA strands, post-amplication, including illustrations of a homogeneous population of long fragments (FIG. 7A), a heterogeneous population of long and short fragments (FIG. 7B), and a homogeneous population of short fragments (FIG. 7C).

### DETAILED DESCRIPTION

By way of overview, the present invention is directed to methods for diagnosing viral infections and determining a status of the infection, including transmissibility of the infection. The invention provides tailored diagnosis relevant to the health status of an individual patient, allowing clinicians to clear patients who have had an infection well before conventional molecular assays would indicate clearance. The invention provides for obtaining a sample from a patient that may contain viral RNA. For example, the patient may be currently exhibiting symptoms of a suspected viral infection or may have come into contact with one or more individuals that either have tested positive for a viral infection or are suspected of having a viral infection.

If it is determined that viral RNA is present, then the patient is identified as testing positive for the viral infection. In this instance, it may be possible that the patient is not exhibiting any signs or symptoms associated with the infection, but could still be contagious. Accordingly, the method of the present invention further allows for the determination of the transmissibility of the viral infection. In particular, the invention takes advantage of the fact that, as the immune system clears a viral infection, viral RNA is degraded. The degraded RNA fragments that are produced as a result of viral neutralization by the immune system are characteristically smaller than fragments obtained from an active viral infection. The invention takes advantage of that insight in order to produce a sensitive and specific diagnostic that distinguishes between patients who have been infected and are producing intact virus (even though they may be asymptomatic), and thus are contagious, and patients who have been infected but are not longer producing active virus and, therefore, are not contagious with respect to the virus. For example, if present, the viral RNA is reverse-transcribed to cDNA and the cDNA is amplified and fragment length is determined.

In some embodiments, cDNA may be amplified using a plurality of primer pairs that are targeted to different regions along a contiguous length of the cDNA. Each primer pair (forward and reverse) will amplify through the length of cDNA that is present from a 5′ end to a 3′ end. Because the primer pairs are arrayed along a contiguous cDNA fragment (i.e., they are targeted against regions of the viral cDNA known to be contiguous), each primer pair will produce an amplicon of equal length if the viral RNA from which the cDNA was derived was intact. If the viral RNA has been degraded, most of the primer pairs will produce no amplicon and any amplicon that is produced will be substantially shorter than the full-length amplicon expected from the intact RNA-derived cDNA. Thus, by simply assessing whether pairs of primers produce amplicons of substantially equal length, it is possible to diagnose an active infection that is still in the virulent state, and thereby make a determination that the patient remains contagious with respect to the virus.

It should be noted that the methods described herein may be used to diagnose a variety of contagious diseases, including microbial and viral. However, for the sake of simplicity and ease of description, the following describes methods for diagnosing and assessing transmissibility of SARS-CoV-2.

SARS-CoV-2 is a virus recently identified as the cause of an outbreak of respiratory illness (referred to as coronavirus disease 2019 (COVID-19)) with an increasing number of patients with severe symptoms and deaths. Typically, with most respiratory viruses, people are thought to be most contagious when they are most symptomatic (the sickest). With SARS-CoV-2, however, there have been reports of spread from an infected patient with no symptoms. Accordingly, to monitor the presence of SARS-CoV-2 and to prevent its spread, it is crucial to detect infection as early and as fast as possible, and further determine transmissibility. The methods of the present invention provide both detection of a viral infection (i.e., presence of the virus in a patient), as well as determination of the status of the infection (i.e., whether a patient is contagious or not).

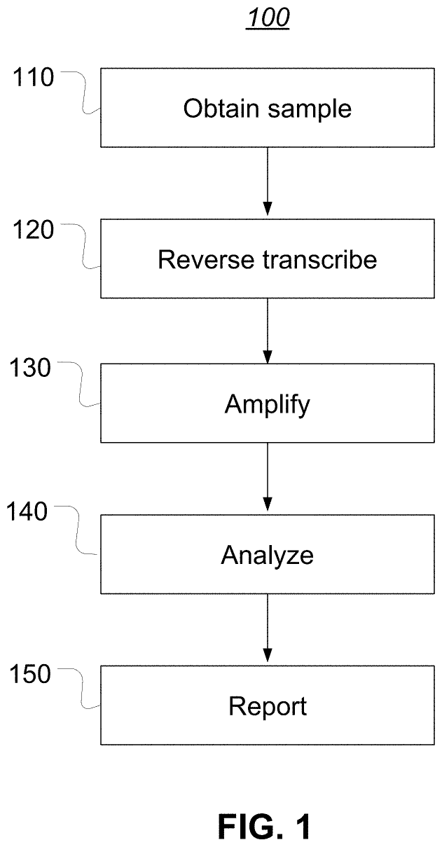

FIG. 1 diagrams a method 100 of assessing transmissibility of a virus. The method 100 includes obtaining 105 a biological sample from a patient. The method of sample collection, as well as the type of sample collected, may be dependent on the specific viral disease to be tested. For example, the biological sample may include a human bodily fluid and may be collected in any clinically acceptable manner. The bodily fluid is generally collected from a patient either exhibiting signs or symptoms of a viral disease, or suspected of having contracted the viral disease due to interaction with others that have tested positive for the disease.

A body fluid may be a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CS. A sample also may be media containing cells or biological material. A sample may also be a blood clot, for example, a blood clot that has been obtained from whole blood after the serum has been removed. In certain embodiments, the sample is blood, saliva, or semen collected from the subject.

For SARS-CoV-2, a biological sample is generally collected via a nasal or throat swab, or, in some cases, saliva. The method 100 includes obtaining a sample of nucleic acids from the biological sample, most notably a isolating a target nucleic acid (i.e., viral RNA) present in the sample. In this instance, the biological sample will be processed to isolate viral RNA associated with SARS-CoV-2. In order to isolate viral RNA, the method includes extracting nucleic acid from the biological sample.

Isolation, extraction or derivation of nucleic acids may be performed by methods known in the art. Isolating nucleic acid from a biological sample generally includes treating a biological sample in such a manner that genomic nucleic acids present in the sample are extracted and made available for analysis. Generally, nucleic acids are extracted using techniques such as those described in Green & Sambrook, 2012, Molecular Cloning: A Laboratory Manual 4 edition, Cold Spring Harbor Laboratory Press, Cold Spring Harbor, N.Y. (2028 pages), the contents of which are incorporated by reference herein. A kit may be used to extract DNA from tissues and bodily fluids and certain such kits are commercially available from, for example, BD Biosciences Clontech (Palo Alto, Calif.), Epicentre Technologies (Madison, Wis.), Gentra Systems, Inc. (Minneapolis, Minn.), and Qiagen Inc. (Valencia, Calif.). User guides that describe protocols are usually included in such kits.

It may be useful to lyse cells to isolate genomic nucleic acid. Cellular extracts can be subjected to other steps to drive nucleic acid isolation toward completion by, e.g., differential precipitation, column chromatography, extraction with organic solvents, filtration, centrifugation, others, or any combination thereof. The genomic nucleic acid may be re-suspended in a solution or buffer such as water, Tris buffers, or other buffers. In certain embodiments the genomic nucleic acid can be re-suspended in Qiagen DNA hydration solution, or other Tris-based buffer of a pH of around 7.5. Isolated nucleic acid (e.g., DNA, RNA, cDNA, etc.) may be fragmented for enhanced probe capture. Methods of nucleic acid fragmentation are known in the art and include, but are not limited to, DNase digestion, sonication, mechanical shearing, and the like. U.S. Pub 2005/0112590 provides a general overview of various methods of fragmenting known in the art. Fragmentation of nucleic acid target is discussed in U.S. Pub. 2013/0274146. The nucleic acid can also be sheared via nebulization, hydro-shearing, sonication, or others. See U.S. Pat. Nos. 6,719,449; 6,948,843; and 6,235,501.

When there is an insufficient amount of nucleic acid for analysis, a common technique used to increase the amount by amplifying the nucleic acid. Amplification refers to production of additional copies of a nucleic acid sequence and is generally carried out using polymerase chain reaction or other technologies well known in the art (e.g., Dieffenbach, PCR Primer, a Laboratory Manual, 1995, Cold Spring Harbor Press, Plainview, N.Y.). Polymerase chain reaction (PCR) refers to methods by K. B. Mullis (U.S. Pat. Nos. 4,683,195 and 4,683,202, hereby incorporated by reference) for increasing concentration of a segment of a target sequence in a mixture of genomic DNA without cloning or purification. Primers can be prepared by a variety of methods including but not limited to cloning of appropriate sequences and direct chemical synthesis using methods well known in the art (Narang et al., Methods Enzymol., 68:90 (1979); Brown et al., Methods Enzymol., 68:109 (1979)). Primers can also be obtained from commercial sources such as Operon Technologies, Amersham Pharmacia Biotech, Sigma, and Life Technologies. Amplification or sequencing adapters or barcodes, or a combination thereof, may be attached to the fragmented nucleic acid. Such molecules may be commercially obtained, such as from Integrated DNA Technologies (Coralville, Iowa). In certain embodiments, such sequences are attached to the template nucleic acid molecule with an enzyme such as a ligase. Suitable ligases include T4 DNA ligase and T4 RNA ligase, available commercially from New England Biolabs (Ipswich, Mass.). The ligation may be blunt ended or via use of complementary overhanging ends.

For example, the method 100 further includes synthesizing DNA from the viral RNA, via reverse transcription 120, to thereby produce complementary DNA (cDNA). As generally understood, reverse transcriptases (RTs) use an RNA template and a short primer complementary to the 3′ end of the RNA to direct the synthesis of the first strand cDNA, which can be used directly as a template for amplification (via PCR). This combination of reverse transcription and PCR (RT-PCR) allows the detection of low abundance RNAs in a sample, and production of the corresponding cDNA, thereby facilitating the cloning of low copy genes. Alternatively, the first-strand cDNA can be made double-stranded using DNA Polymerase I and DNA Ligase. Many RTs are available from commercial suppliers. The use of engineered RTs improves the efficiency of full-length product formation, ensuring the copying of the 5′ end of the mRNA transcript is complete, and enabling the propagation and characterization of a faithful DNA copy of an RNA sequence. The use of the more thermostable RTs, where reactions are performed at higher temperatures, can be very helpful when dealing with RNA that contains high amounts of secondary structure.

The method 100 further includes amplifying 130 the cDNA. In preferred embodiments, the target nucleic acid is amplified using by a PCR reaction. For example, amplification may be performed using any one of real-time PCR (quantitative PCR or qPCR), reverse-transcriptase (RT-PCR), multiplex PCR, nested PCR, high-fidelity PCR, fast PCR, hot start PCR, GC-rich PCR, or any similar technique using a polymerase to synthesize a nucleic acid.

The amplification process 130 results in production of amplicons. Ideally, the cDNA is amplified using a plurality of primer pairs that are targeted to different regions along a contiguous length of the cDNA. Each primer pair (forward and reverse) will amplify through the length of cDNA that is present from a 5′ end to a 3′ end. The method 100 further includes analyzing 140 data from the amplification step 130 to determine the transmissibility of the virus. In particular, the primer pairs used in the amplification step 130 are arrayed along a contiguous cDNA fragment (i.e., they are targeted against regions of the viral cDNA known to be contiguous). As a result, each primer pair will produce an amplicon of equal length if the viral RNA from which the cDNA was derived was intact. If the viral RNA has been degraded, most of the primer pairs will produce no amplicon and any amplicon that is produced will be substantially shorter than the full-length amplicon expected from the intact RNA-derived cDNA. Accordingly, analysis 140 of the amplification data (i.e., amplicons) generally includes determining a length of amplicons produced in the amplifying step and further determining the transmissibility of the virus based on analysis of the determined lengths of amplicons.

In particular, a virus may be determined as being transmissible (i.e., contagious) if the length of amplicons is greater than a predetermined threshold length and determined as not being transmissible (i.e., non-contagious) if the length of amplicons is less than a predetermined threshold length. The predetermined threshold length may be based, at least in part, on a known length (i.e., approximate base pair length) at which the virus is transmissible, which may be based on continual studies of infected patients and their respective transmission rates at a particular stage of the disease. Additionally, or alternatively, transmissibility of the virus may be based on a determination of whether the primer sets produce amplicons of substantially identical length. In other words, by amplifying the cDNA using a plurality of primer sets arrayed along a contiguous length of the cDNA, each primer pair will produce an amplicon of equal length if the viral RNA from which the cDNA was derived was intact. Accordingly, the virus may be determined as being transmissible (i.e., contagious) if the amplicons are of substantially identical length and determined as not being transmissible (i.e., non-contagious) if the amplicons are not of substantially identical length. Again, if the viral RNA has been degraded, most of the primer pairs will produce no amplicon and any amplicon that is produced will be substantially shorter than the full-length amplicon expected from the intact RNA-derived cDNA.

In some embodiments, methods of the present invention utilize quantitative PCR (qPCR) in order to provide relative quantities of amplicons. By knowing relative quantities of amplicons, subsequent quantitative analysis can be performed for the determination of whether a given sample is exhibiting an active (i.e., transmissible) viral infection or a cleared viral infection. In particular, as a viral infection is cleared by the immune system or therapy, the number of intact (long) fragments, indicative of an active viral infection, decreases and the number of shorter fragments, indicative of viral neutralization, increases. Accordingly, during an active infection, primer pairs will produce fragments of equivalent length, whereas, upon neutralization, fragments will be smaller. As such, the greater the number of smaller fragments, the greater the likelihood that the viral infection is cleared and the patient can be deemed to be non-contagious.

The method 100 further includes providing 150 a report comprising information related to virus evaluation, including, specific data associated with the assay, whether the sample tested positive or negative for the virus, and, if positive, a determination of the transmissibility of the virus.

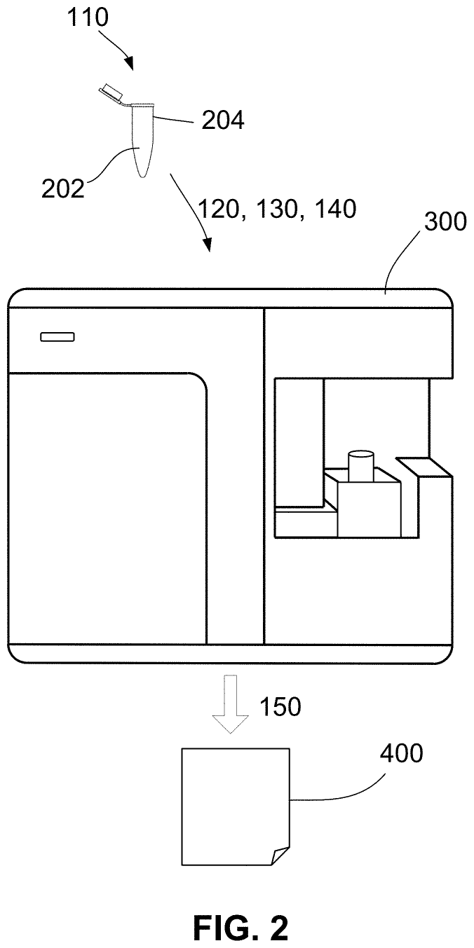

FIG. 2 shows a sample 202 within a suitable container 204 that is obtained 110 from a patient suspected of having a viral infection. For example, in some embodiments, samples may be collected and stored in their own container, such as a centrifuge tube such as the 1.5 mL micro-centrifuge tube sold under the trademark EPPENDORF FLEX-TUBES by Eppendorf, Inc. (Enfield, Conn.). FIG. 2 further illustrates loading of the sample 202 into an instrument 300 capable of performing one or more assays on the sample 202 to determine whether viral RNA associated with the virus is present and to further determine the transmissibility of the viral infection. In particular, the instrument 300 may be configured to provide any one of the prior steps of method 100, including, but not limited to, detection of viral RNA, reverse transcribing of RNA to produce cDNA (operation 120), amplification of cDNA (operation 130), analysis of data from the amplification step (operation 140), and generation of a report 400 providing information related to the virus evaluation (operation 150). Accordingly, the instrument 300 is generally configured to detect, sequence, and/or count the target nucleic acid(s) or resulting fragments. In this instance, where a plurality of fragments are present or expected, the fragment may be quantified, e.g., by qPCR. The resulting report 400 may include the specific data associated with the assay, including, for example, patient data (i.e., background information, attributes and characteristics, medical history, tracing information, etc.), test data, including whether the sample tested positive or negative for the virus, and, if positive, a determination of the transmissibility of the virus.

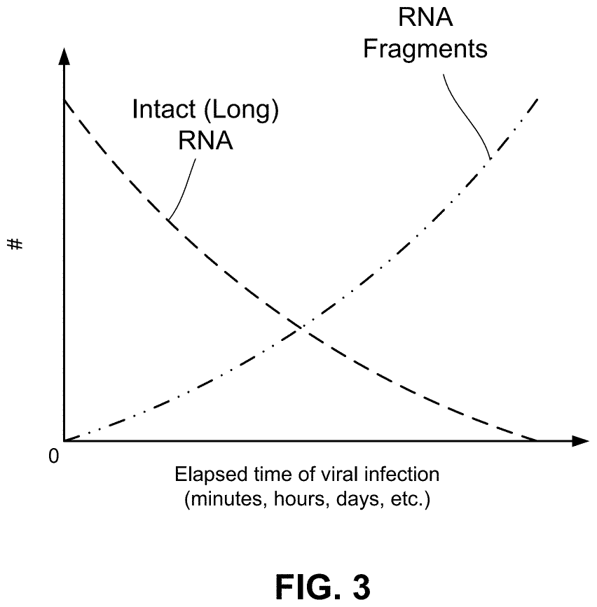

FIG. 3 is a graph illustrating a process of viral clearance resulting in fragmentation of viral DNA. As previously described, the invention takes advantage of the fact that, as the immune system clears a viral infection, viral RNA is degraded. In particular, as shown in the graph, the number of intact (long) viral fragments, indicative of an active viral infection, decreases and the number of shorter viral fragments, indicative of viral neutralization, increases as a viral infection is cleared by the immune system or therapy. The degraded RNA fragments that are produced as a result of viral neutralization by the immune system are characteristically smaller than fragments obtained from an active viral infection. The invention takes advantage of that insight in order to produce a sensitive and specific diagnostic that distinguishes between patients who have been infected and are producing intact virus (even though they may be asymptomatic), and thus are contagious, and patients who have been infected but are not longer producing active virus and, therefore, are not contagious with respect to the virus. Accordingly, during an active infection, primer pairs will produce fragments of equivalent length, whereas, upon neutralization, fragments will be smaller. As such, the greater the number of smaller fragments, the greater the likelihood that the viral infection is cleared and the patient can be deemed to be non-contagious.

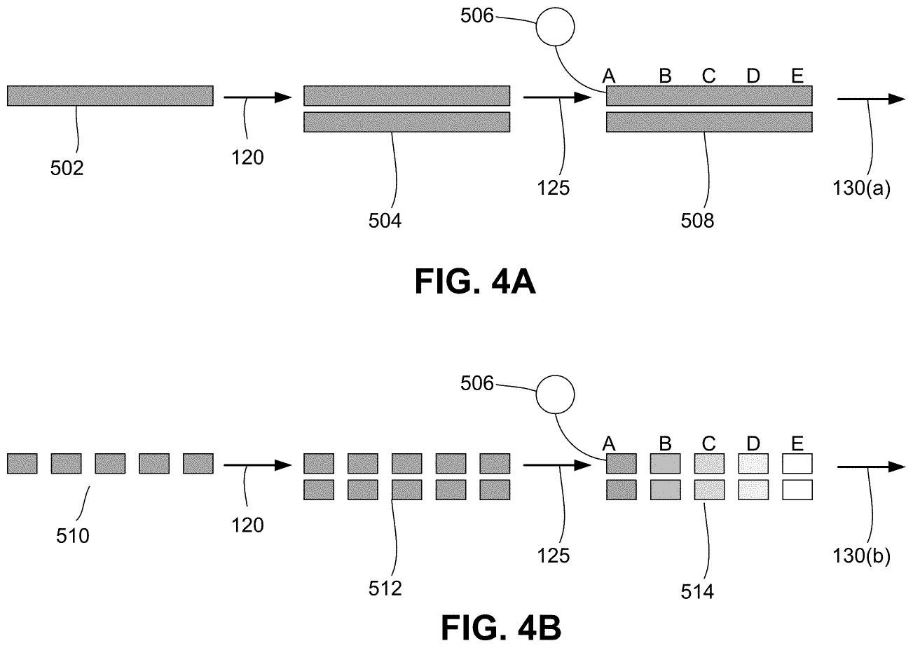

FIGS. 4A and 4B illustrate operations of the transmissibility assessment method on a full-length viral RNA strand 502 and a fragmented RNA strand 510, respectively. The methods of the present invention are able to distinguish between an active (i.e., transmissible) viral infection and an inactive (i.e., non-transmissible) viral infection based, at least in part, on a determination of fragment length of processed viral RNA.

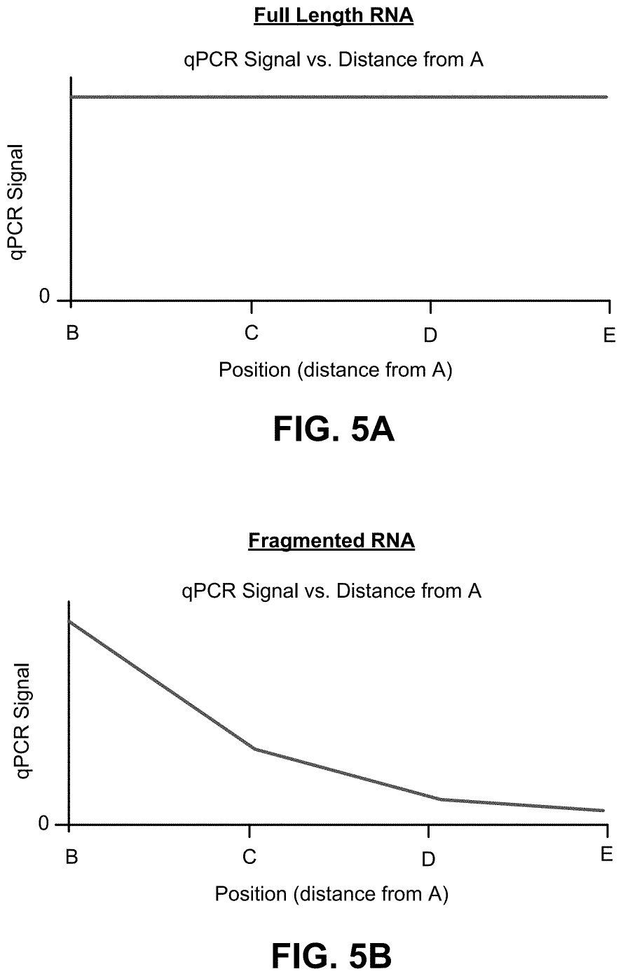

Referring to FIG. 4A, upon isolating viral RNA from a biological sample, which may include a full-length viral RNA strand 502, the RNA is reverse-transcribed (operation 120) to cDNA 504. The cDNA 504 is amplified and fragment length 508 is determined (via quantitative analysis). For example, the cDNA 504 may be amplified 130(a) using a plurality of primer pairs 506 that are targeted to different regions along a contiguous length of the cDNA. Each primer pair (forward and reverse) will amplify through the length of cDNA that is present from a 5′ end to a 3′ end. Because the primer pairs 506 are arrayed along a contiguous cDNA fragment (i.e., they are targeted against regions (e.g., A, B, C, D, E) of the viral cDNA 508 known to be contiguous), each primer pair will produce an amplicon of equal length if the viral RNA from which the cDNA was derived was intact, as illustrated in the graph of FIG. 5A. In particular, FIG. 5A is a graph illustrating quantitative analysis of the full-length RNA strand 502 processed in accordance with the method described herein and having undergone qPCR (operation 130(a)). As illustrated, during an active infection, primer pairs will produce fragments of equivalent length. Accordingly, in this instance, the patient will be deemed as testing positive, as well as producing intact virus (even though they may be asymptomatic), and thus are contagious,

Referring to FIG. 4B, upon isolating viral RNA from a biological sample, which may include a fragmented viral RNA strand 510, the RNA is reverse-transcribed (operation 120) to cDNA 512. The cDNA 512 is amplified and fragment length 514 is determined (via quantitative analysis). For example, the cDNA 514 may be amplified 130(b) using a plurality of primer pairs 506 that are targeted to different regions along a contiguous length of the cDNA. Each primer pair (forward and reverse) will amplify through the length of cDNA that is present from a 5′ end to a 3′ end. If the viral RNA has been degraded, which, in this case, it has been, most of the primer pairs will produce no amplicon and any amplicon that is produced will be substantially shorter than the full-length amplicon expected from the intact RNA-derived cDNA, as illustrated in FIG. 5B. In particular, FIG. 5B is a graph illustrating quantitative analysis of the fragmented RNA strand 510 processed in accordance with the method described herein and having undergone qPCR (operation 130(b)). As illustrated, upon neutralization of a viral infection, fragments are smaller and thus are not of equal length, thereby indicating viral clearance. Accordingly, in this instance, the patient will be deemed as testing positive, but are no longer producing active virus and, therefore, are not contagious with respect to the virus.

It should be noted that positive selection of the targeted regions of a cDNA fragment may include, but are not limited to, the use of biotinylated oligomer hybridization, and biotinylated sequence specific RT-primer(s).

### EXAMPLES

While conventional RT-PCR protocols are able to detect the presence of viral infection, and thereby determine whether a patient is positive, such protocols lack the ability to determine whether individuals who have been, or are currently, infected with COVID-19, are still infectious during their recovery.

The basis for the difficulty in determining a patient's infectious state is the fact that RT-PCR protocols used as diagnostic tests are based on small targets in order to maximize analytical sensitivity and maximize clinical sensitivity and specificity. By only performing small amplicon amplification, RT-PCR assays of the prior art are unable to distinguish between individuals who are no longer infectious, but continue to generate non-viable small fragments of the SARS-CoV-2 genome; and those patients who are still infectious and are shedding viable virus representing an intact viral genome (e.g., ˜30 kb). Furthermore, the SARS-CoV-2 strain of coronavirus appears to have a unique clinical presentation and pathway in different individuals. Therefore, it is difficult to determine when a patient is able to return to social interaction without the concern of infecting others. Alternatively, long RT-PCR assays could be performed on viral targets purified from patients, but the lack of robustness associated with long RT-PCR can compromise analytical results, especially in situations where there is the chance that very little of the target is provided in a given sample, thereby further reducing the possibility of detecting smaller fragments. In addition, attempting to perform long and short amplicon amplification may also introduce bias toward the shorter amplicon amplification and potentially lead to false negative results on individuals who may have a small number of viable virus and still be infectious.

Methods of the present invention utilize a combination of validated techniques to isolate and detect the presence of viral RNA (associated with SARS-CoV-2) and further determine a status of the infection, including transmissibility of the infection. In particular, preferred methods of the present invention provide for a coronavirus targeted specific enrichment of reverse transcription products with short, efficient, and robust PCR amplification reactions positioned at various locations distant from the origin of sequence specific capture that enables the efficiency and maximum sensitivity of utilizing short PCR amplification reactions, while determining and quantifying ratios of long versus short viral targets for determining a status of a patient along the continuum of infection, including a determination of whether the patient is no longer infectious. This methodology allows a tailored and personalized diagnosis relevant to the health status of an individual patient, including an infectious determination of any given patient regardless of their personal immune response to the COVID-19 infection, and allows them to return to social contact without the concern of infecting others.

The following provides an example of an experimental approach of assessing transmissibility of a virus, notably SARS-CoV-2 in accordance with methods of the present invention. A biological sample is obtained from a patient. The method of sample collection, as well as the type of sample collected, may be dependent on the specific viral disease to be tested. For example, the biological sample may include a human bodily fluid and may be collected in any clinically acceptable manner. The bodily fluid is generally collected from a patient either exhibiting signs or symptoms of a viral disease, or suspected of having contracted the viral disease due to interaction with others that have tested positive for the disease.

For SARS-CoV-2, a biological sample is generally collected via a nasal or throat swab, or, in some cases, saliva. In other examples, the sample may include an aerosol sample or droplets obtained in air or, more preferably, via the expulsion of droplets with a cough or sneeze.

### RNA Sample Preparation:

Upon collecting a biological sample, a target nucleic acid (i.e., viral RNA) is isolated. RNA can be purified from patient samples by utilizing standard RNA prep. Such methods and kits, may include, but are not limited to, QIAmp RNA preparation kits, EZ1 virus preparation kits for automated RNA preparation, as well as similar kits from Thermo Fisher (e.g., MagMax extraction kits).

RNA Conversion into cDNA:

The methods further include synthesizing DNA from the viral RNA, via reverse transcription, to thereby produce complementary DNA (cDNA). It should be noted that standard cDNA kits can also be utilized to generate Coronavirus specific cDNA from the RNA purified from the patient. Kits such as iScript cDNA kit sold by BioRad that can efficiently make cDNA greater than 8 kb in length. In addition, other cDNA kits are readily available from Thermo Fisher, Takara, Invitrogen, and others.

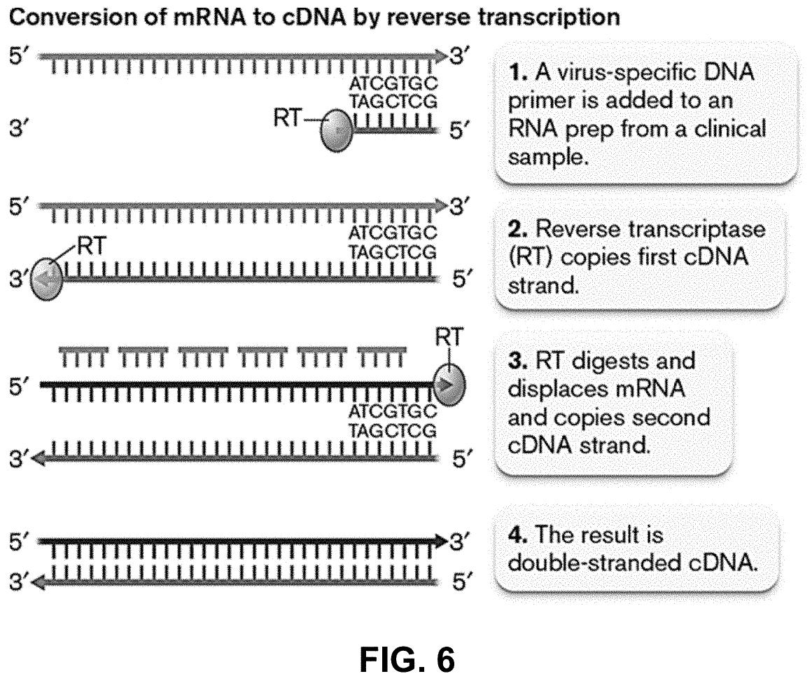

FIG. 6 illustrates operations of converting mRNA to cDNA via reverse transcription in accordance with the methods of the present invention. Viral RNA, if present, is extracted from the sample and reverse-transcribed into cDNA with primer pairs comprising a binding member. The cDNA is then amplified and fragment length is determined for subsequent determination of transmissibility. The binding member can be any convenient moiety (e.g., haptenated or biotinylated). The amplicons are bound to a solid support via the binding member and a transmissible infection is identified as a number of amplicons exceeding a predetermined threshold length. The amplicons are preferably labeled for detection (e.g., with a colorimetric label or other identifiable marker). Ideally, cDNA is amplified using a plurality of primer pairs that are targeted to different regions along a contiguous length of the cDNA. In one embodiment, the conversion of mRNA to cDNA via reverse transcription may include the use of a modified oligonucleotide (e.g., Biotin added to the 5′ end of the oligo) that is used to prime the 3′ end of the RNA target and extended via reverse transcriptase to generate the cDNA strand. Because the primer pairs are arrayed along a contiguous cDNA fragment (i.e., they are targeted against regions of the viral cDNA known to be contiguous), each primer pair will produce an amplicon of equal length if the viral RNA from which the cDNA was derived was intact. If the viral RNA has been degraded, most of the primer pairs will produce no amplicon and any amplicon that is produced will be substantially shorter than the full-length amplicon expected from the intact RNA-derived cDNA. Thus, by simply assessing whether pairs of primers produce amplicons of substantially equal length, it is possible to diagnose an active infection that is still in the virulent state, and thereby make a determination that the patient remains contagious with respect to the virus.

In one embodiment, the primers are directed to selected regions of the cDNA along the length of a reverse-transcribed cDNA strand from one end to the other, with different colors representing the series of tiled primers. Thus, colorimetric detection can be accomplished simply by observing (and possibly quantifying) color. For example, an intact (full-length or near full- length) sequence will result in a multi-color output (indicating a transmissible virus); whereas a single color or a predomination of a single color, indicates RNA that has been degraded as a result of the immune response to infection, indicating a less-transmissible infection.

### Transmissibility Determination (Quantitative Analysis):

In some embodiments, methods of the present invention utilize quantitative PCR (qPCR) in order to provide relative quantities of amplicons. By knowing relative quantities of amplicons, subsequent quantitative analysis can be performed for the determination of whether a given sample is exhibiting an active (i.e., transmissible) viral infection or a cleared viral infection. In particular, as a viral infection is cleared by the immune system or therapy, the number of intact (long) fragments, indicative of an active viral infection, decreases and the number of shorter fragments, indicative of viral neutralization, increases. Accordingly, during an active infection, primer pairs will produce fragments of equivalent length, whereas, upon neutralization, fragments will be smaller. As such, the greater the number of smaller fragments, the greater the likelihood that the viral infection is cleared and the patient can be deemed to be non-contagious.

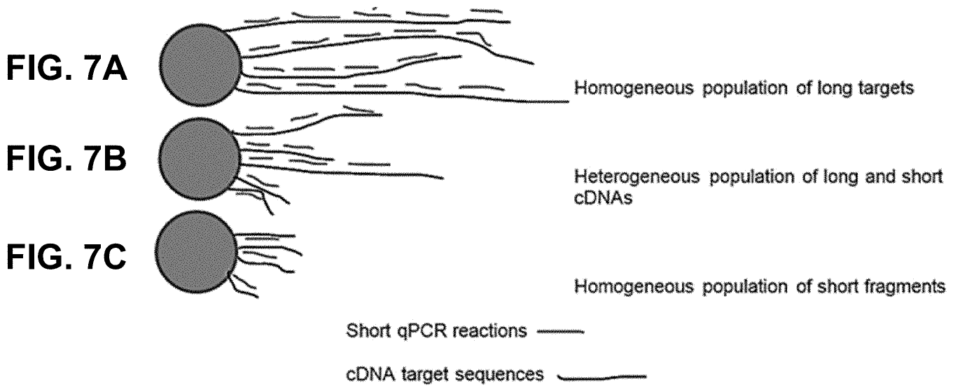

FIGS. 7A, 7B, and 7C show representative illustrations of populations of resulting RNA strands, post-amplication, including illustrations of a homogeneous population of long fragments (FIG. 7A), a heterogeneous population of long and short fragments (FIG. 7B), and a homogeneous population of short fragments (FIG. 7C). As previously described, qPCR reactions can be performed separately or as a multiplex to determine the quantity of the different length products. The ratios of long versus targets can be associated with clinical/infectious status, as described herein,

It should be noted that in some embodiments, the cDNA products are captured on beads via hybrid capture procedures, such that unbound material is washed away and the bound material is released to be analyzed in single molecule analytical platforms (e.g., BioRad, 10×, Quanterix, etc.). Under these conditions of analysis, a multiplex PCR reaction is carried out in the presence of a single molecule template. Accordingly, results of such an analysis may simply include a matter of counting of how many molecules generate a PCR reaction product in each well or how many molecules demonstrate multiple reactions occurring in a single well. This single molecule approach would yield much higher resolution of quantification.

Accordingly, the present invention improves on conventional molecular viral detection by differentiating between intact viral RNA indicative of an active (and transmissible) infection and fragmented RNA indicative of viral clearance. In addition, the invention allows for quantifying an amount of RNA suspected to be associated with an active (i.e., transmissible) viral infection and/or quantifying RNA fragments associated with a cleared viral infection. The invention takes advantage of the fact that, as the immune system clears a viral infection, viral RNA is degraded. The degraded RNA fragments that are produced as a result of viral neutralization by the immune system are characteristically smaller than fragments obtained from an active viral infection. The invention takes advantage of that insight in order to produce a sensitive and specific diagnostic that distinguishes between patients who have been infected and are producing intact virus (even though they may be asymptomatic), and thus are contagious, and patients who have been infected but are not longer producing active virus and, therefore, are not contagious with respect to the virus.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of assessing transmissibility of a virus, the method comprising the steps of:
reverse transcribing cDNA from viral RNA in a biological sample;
amplifying said cDNA to produce amplicons; and
identifying a virus as transmissible if said amplicons are greater than a predetermined threshold length.

2. The method of claim 1, further comprising identifying a virus as non-transmissible if said amplicons are less than a predetermined threshold length.

3. The method of claim 1, wherein the predetermined threshold length is based, at least in part, on a known length associated with positive transmission of the virus from an infected subject to a non-infected subject.

4. The method of claim 3, wherein the known length is based on a positive correlation between a rate of transmission and stage of viral clearance.

5. The method of claim 1, wherein said amplifying step comprises using a plurality of primer pairs that are targeted to different regions along a contiguous length of said cDNA.

6. The method of claim 1, wherein the biological sample comprises a bodily fluid.

7. The method of claim 6, wherein the bodily fluid comprises mucus and/or saliva.

8. The method of claim 7, further comprising the step of obtaining the biological sample via a nasal or throat swab.

9. The method of claim 1, wherein the virus comprises a coronavirus.

10. The method of claim 9, wherein the coronavirus is severe acute respiratory syndrome coronavirus-2 (SARS-CoV-2).

11. A method of identifying an active viral infection, the method comprising the steps of:
reverse transcribing cDNA from viral RNA in a biological sample;
amplifying said cDNA using a plurality of primer sets arrayed along a contiguous length of the cDNA;
determining whether said primer sets produce amplicons of substantially identical length; and
identifying an active viral infection if said amplicons are of substantially identical length.

12. The method of claim 11, further comprising identifying an inactive viral infection if said amplicons are not of substantial identical length.

13. The method of claim 11, wherein the plurality of primer pairs are targeted to different regions along a contiguous length of said cDNA.

14. The method of claim 13, wherein the different regions are associated with regions of the viral cDNA known to be contiguous.

15. The method of claim 11, wherein the biological sample is obtained from a subject suspected of having the viral infection.

16. The method of claim 11, wherein the biological sample comprises a bodily fluid.

17. The method of claim 16, wherein the bodily fluid comprises mucus and/or saliva.

18. The method of claim 17, further comprising the step of obtaining the biological sample via a nasal or throat swab.

19. The method of claim 1, wherein the virus comprises a coronavirus.

20. The method of claim 19, wherein the coronavirus is severe acute respiratory syndrome coronavirus-2 (SARS-CoV-2).
